(19) United States
(12) Patent Application Publication
(10) Pub. No.: US 2014/0332693 A1
(43) Pub. Date: Nov. 13, 2014

(54) METHOD OF USING THE NOVEL POLYMER GEL FOR MAGNETIC RESONANCE IMAGING (MRI) VALIDATION

(71) Applicants: KING FAISAL SPECIALIST HOSPITAL AND RESEARCH CENTRE, RIYADH (SA); King Abdulaziz City for Science and Technology, RIYADH (SA)

(72) Inventors: AHMED ALI BASFAR, RIYADH (SA); Belal Moftah, RIYADH (SA); KHALID A RABAEH, RIYADH (JO); Akram A. Almousa, RIYADH (SA)

(21) Appl. No.: 14/194,975

(22) Filed: Mar. 3, 2014

Related U.S. Application Data
(63) Continuation-in-part of application No. 13/892,484, filed on May 13, 2013.

(51) Int. Cl. A61K 49/06; G01T 1/02

(52) U.S. Cl. CPC A61K 49/06; G01T 1/02
USPC 250/393; 424/9.3

(57) ABSTRACT

Novel normoxic N-(Hydroxymethyl) acrylamide (NHMAGAT) polymer gel dosimeters for MRI scanning are introduced for radiotherapy planning system. The composition of the NHMAGAT polymer gel and method of making the NHMAGAT polymer gel dosimeter are disclosed. The dosimeters are irradiated with Varian Rapid Arc linear systems at different absorbed doses. The results show that the percent depth dose and 2D plan dose distribution of NHMAGAT polymer gel dosimeters are in a good agreement with the ionization chamber measurements and CT planned dose distribution, respectively.

### CROSS REFERENCE TO RELATED APPLICATIONS

The instant application is a Continuation in part of a U.S. application Ser. No. 13/892,484, filed on May 13, 2013. The pending U.S. application Ser. No. 13/892,484 is hereby incorporated by reference in its entireties for all of its teachings.

### FIELD OF INVENTION

The present disclosure describes a novel composition of a polymer gel and validating it for using in magnetic resonance imaging (MRI). More specifically it relates to a dosimetry validation of a novel polymer gel dosimeters containing N-(Hydroxymethyl) acrylamide in conjunction with MRI.

### BACKGROUND

Advanced treatment techniques such as intensity modulated radiotherapy (IMRT), brachytherapy and stereotactic radio-surgery (SRS) and boron nuclear capture therapy (BNCT) require highly conformal 3D dose distributions. Polymer gel dosimeters have shown to be a very useful tool in the verification of radiotherapy treatment planning system (TPS), as it offers a number of advantages over traditional single point-dose dosimeters such as ionization chambers and thermo-luminescent dosimeters (TLD) and two-dimensional (2D) radiographic films. The advantages include independence of radiation direction, radiological soft tissue equivalence, integration of dose for a number of sequential treatment fields, and perhaps most significantly, evaluation of a complete tumor volume at one time (De Deene, Y. 2009). MRI was suggested as the first method to measure 3D complex dose distributions produced by ionizing radiation absorbed in tissue-equivalent (TE) gelatin by Gore, J. C. et. al., 1984.

The first proposed TE gel was the chemical solution of ferrous sulfate (Fricke) dosimeter. Upon irradiation, radiation-induced free radicals produced by the radiolysis of water oxidize ferrous ions (Fe2+) to ferric ions (Fe3+) that alters the magnetic movement of the metal ion. As a result, the spin-spin relaxation times of water protons in the aqueous gel are reduced and consequently increase the nuclear magnetic resonance (NMR) spin-spin relaxation rate (R2) of the protons (De Deene, Y. et. al. (2006). A major limitation in Fricke gel systems is the diffusion of ions Fe3+within the dosimeter over time, resulting in the change of dose distribution with time i.e. MRI scans must be taken immediately after irradiation.

However, none of the above-discussed references disclose or suggest a relatively inexpensive but highly effective 3D polymer composition for MRI use. Accordingly, there exists a need to overcome the deficiencies and limitations described herein above.

### SUMMARY OF THE INVENTION

The present invention relates to polymer gel dosimeters containing N-(Hydroxymethyl) acrylamide (NHMA).These polymer gels are tissue equivalent polymer gels whose nuclear magnetic resonance (NMR) and optical properties change with radiation dose. They may provide unique advantages for measuring radiation dose distributions in three-dimensional (3D).

In one embodiment, polymer gel dosimeters were made by polymerizing of N-(Hydroxymethyl) acrylamide (NHMA) under a fume hood in normal atmospheric conditions (NHMAGAT gel). In one embodiment the NHMAGAT was studied as a novel composition for polymer gel dosimeters to be used for radiotherapy treatment planning system. In another embodiment, the polymerized gels were irradiated with (Varian Rapid Arc linear systems, USA) at different absorbed doses. In another embodiment, Magnetic resonance imaging (MRI) scanning was used to calculate transverse relaxation rate R₂. The change in R₂ corresponding to the amount of polymer formation in polymer gel dosimeters increases gradually with absorbed dose. It was found that the percent depth dose and 2D plan dose distribution of NHMAGAT polymer gel dosimeters are in a good agreement with the ionization chamber measurements and computerized tomography (CT) planned dose distribution, respectively.

In one embodiment, the composition of the polymer gel is Gelatin between 2-5 g w/w % by weight, N-(Hydroxymethyl) acrylamide (NHMA) is between 1-12 w/w % by weight, N, N -methylene-bis-acrylamide (BIS) is between 1-4 w/w % by weight, Hydroquinone (HQ) is between 0-0.1 mM, Trakis (hydroxymethyl) phosphonium chloride (THPC) is between 1-50 mM and Ultra-pure de-ionized water as a solvent.

In one embodiment, the process of making and using the NHMAGAT polymer gel dosimeters for MRI scanning involves adding gelatin to the ultra-pure de-ionized water and leaving it to soak for 10 minutes before heating to 50° C. for 1 hour. After the solution has cooled down to 40° C., NHMA and HQ are added, respectively and stirred until completely dissolved. After the solution has cooled down to 35° C., THPC is added as antioxidant. For characterization/validation study, the final polymer gels are filled into calibration tubes, bottle (5 cm diameter, 11 cm height)and container (12.5 cm diameter, 17 cm height), respectively and sealed. All gels are stored in a refrigerator (10° C.) overnight prior to irradiation.

In one embodiment, a method of validating the polymer gel is described. The methods and validation for MRI use disclosed herein may be implemented in any means for achieving various aspects, and may be executed manually or automated using a computer. Other features will be apparent from the accompanying figures and from the detailed description that follows.

### BRIEF DESCRIPTION OF DRAWINGS

Example embodiments are illustrated by way of example in the accompanying figure and like references indicate similar elements and in which:

FIG. 1 shows Percentage depth dose of NHMAGAT polymer gel dosimeter (square) as a function of depth for 8 Gray (Gy) beam source compared with the measurements by ionization chamber (full curve).

FIG. 2a shows 2D NHMAGAT polymer gel dosimeter picture after exposure.

FIG. 2b of NHMAGAT polymer gel dosimeters planned dose distribution at the same depth in gel.

Other features of the present embodiments will be apparent from the accompanying figures and from the detailed description that follows.

### DETAILED DESCRIPTION

Several embodiments for 3D gel composition and method of use are disclosed. Although the present embodiments have been described with reference to specific example embodiments, it will be evident that various modifications and changes may be made to these embodiments without departing from the broader spirit and scope of the various embodiments.

The limitations expressed in the background section led to the investigation of alternative gel dosimetry systems which could be used in conjunction with MRI scans and based on a set of several simple considerations, i.e. the gels must be tissue equivalent and mechanically strong, the monomers must be highly reactive so that a significant amount of the polymer may be formed per unit concentration of free radical initiators produced by water radiolysis. A relatively high cross-linking density of the polymer is clearly needed to produce significant NMR relaxation effect of water protons (Maryanski, et.al. 1997).The 3D gel dosimeters may be used for planning the radiotherapy treatment of patients to evaluate the distribution of absorbed dose in the phenomenon which simulates the human organ to be treated. So, the use of these gels is pertinent for the planning phase of the treatment before exposing the patient to radiotherapy. Several other uses are envisioned such as developing NHMAGAT gel dosimeter for 3D radiotherapy treatment planning using MRI scan and Developing NHMAGAT gel dosimeter for 3D nuclear medicine using gamma camera.

Radiation-induced changes in a polymer gel dosimeter manufactured using 2-hydroxyethylacrylate (HEA) and BIS are investigated using magnetic resonance imaging (MRI) (Gustaysson, H., et.al. 2004). The gels are manufactured in a glove box facility, which are purged with nitrogen at least 18 hours before and continuously during the mixing of the gel. It is observed that the dose response decreases with increasing the concentration of gelatin while it increases with increasing the concentration of co-monomer. Gelatin molecules consume some of the free radicals created in water radiolysis. Thus, a lowered gelatin concentration will lead to an increased number of radicals available for initiation of the polymerization process.

The first normoxic gel, known as MAGIC (methacrylic and ascorbic acid in gelatin initiated by copper (II) sulphate, ascorbic acid and hydroquinone) was made, which can be produced, stored and irradiated in a normal or normoxic environment without purging nitrogen (Fong, P. M. et.al.,2001).

Venning A. J., et.al. (2005) investigated PAGAT polymer gel dosimeter using magnetic resonance imaging. Polyacrylamide gel (PAG) dosimeter consists of co-monomers (AA and BIS) with the anti-oxidant Tetrakis (hydroxymethyl) phosphonium chloride (THPC) and the polymerization inhibitor hydroquinone (HQ) to form the normoxic PAGAT polymer gel dosimeter. PAGAT polymer gel displayed a high degree of spatial stability over the 24 days period, good dose resolution and spatial integrity.

Mariani, M. et. al. (2007) introduced anew measurement technique for polymer gel dosimeter using spectroscopy meter. PAGAT polymer gel dosimeters were used in this study based on the work of Venning A J, et.al.(2005). Dosimeter-gel samples were optically analyzed by measuring the visible-light transmittance through gel samples by an UV-VIS spectrophotometer. It was found that the dose response increased linearly with increase of absorbed dose up to 20 Gy. Recent developments of complex radiotherapy treatment techniques have emphasized the need for a dosimetric system with the ability to measure absorbed dose distributions in three dimensions and with high spatial resolution. Therefore, normal and normoxic polymer gel dosimeters containing N-(Hydroxymethyl) acrylamide are introduced in this invention/disclosure for radiation therapy with low toxicity, low cost and high dose response compared to the commercial product based on PAGAT (Maryanski, M., et.al., 1994).

A polymer gel dosimeter based on radiation-induced polymerization of N-(Hydroxymethyl) acrylamide (NHMA) under a fume hood in normal atmospheric conditions (NHMAGAT) are synthesized and studied as new compositions of polymer gel dosimeters for radiotherapy treatment planning system. The dosimeters are irradiated with (Varian RapidArc linear systems, USA) at different absorbed doses. Magnetic resonance imaging (MRI) scanning is used to calculate transverse relaxation rate R₂. The change in R₂ corresponding to the amount of polymer formation in polymer gel dosimeters increases gradually with absorbed dose. It is found that the percent depth dose and 2D plan dose distribution of NHMAGAT polymer gel dosimeters are in a good agreement with the ionization chamber measurements and CT planned dose distribution, respectively.

### Composition and Method of Making the Polymer Gel:

The normoxic N-(Hydroxymethyl) acrylamide polymer gels (NHMAGAT) are synthesized under a fume hood in normal atmospheric conditions. The NHMAGAT dosimeters are composed of 1-12% w/w % N-(Hydroxymethyl) acrylamide (NHMA) monomer, 1-4% N, N -methylene-bis-acrylamide (BIS) cross-linker, 2-5 g w/w % gelatin (Type A, bloom 300), 0-0.1 mM hydroquinone (HQ) and 1-50 mM tetrakis (hydroxymethyl) phosphonium chloride (THPC). All co-chemicals were obtained from SIGMA Chemical Co. (St. Louis, Mo., USA). In one specific example the NHMAGAT dosimeters are composed of 8 w/w % N-(Hydroxymethyl)acrylamide (NHMA) monomer, 3 w/w % N, N-methylene-bis-acrylamide (BIS) cross-linker, 3 w/w % gelatin (Type A, bloom 300), 0.02 mM hydroquinone (HQ) and 5 mM tetrakis (hydroxymethyl) phosphonium chloride (THPC).

The gelatin is added to the ultra-pure de-ionized water and left to soak for 10 minutes before heating to 50° C. for 1 hour. After the solution is cooled down to 40° C., NHMA, HQ and BIS are added respectively and stirred until completely dissolved. After the solution is cooled down to 35° C., THPC is added as antioxidant. The final polymer gels are filled into calibration tubes, bottle (5 cm diameter, 11 cm height and container (12.5 cm diameter, 17 cm height), respectively and sealed. All polymer gel filled dosimeters are stored in a refrigerator (10° C.) overnight prior to irradiation.

### Validation of the Polymer Gel to be Used for MRI

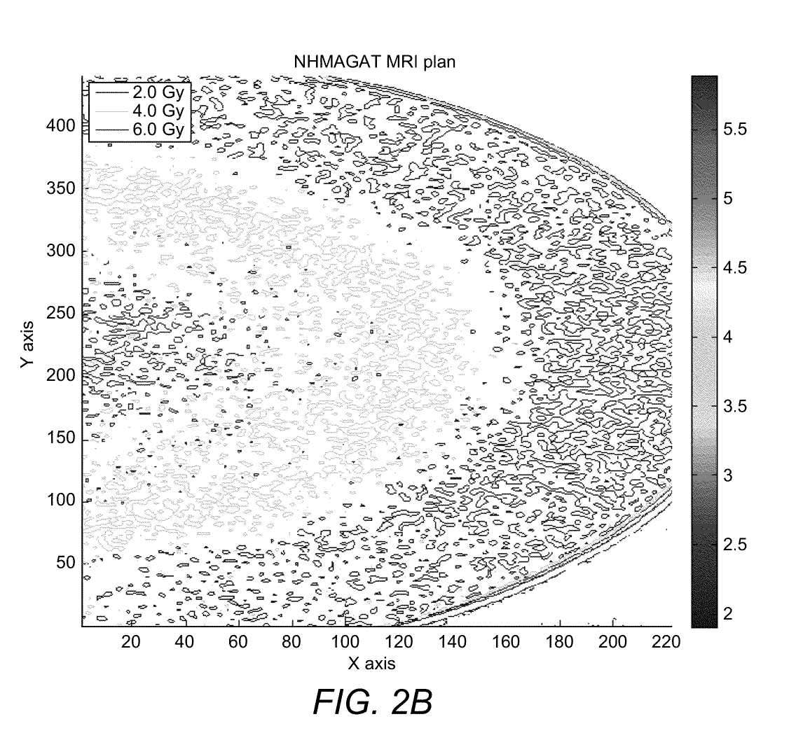

Five hours before irradiation, all polymer gel dosimeters were transferred to a temperature controlled Varian RapidArc linear accelerator machine room to equilibrate to room temperature. All imaging is performed at the ambient air temperature of 21° C. The calibration tubes are irradiated to 2, 4, 6, 8 and 10 Gy while the bottle (5 cm diameter, 11 cm height)and the container (12.5 cm diameter, 17 cm height) are irradiated to 8 Gy for depth dose measurement to computer tomography (CT) scan as shown in FIG. 2 (Left). Five hours before imaging all polymer gel dosimeters are transferred to a temperature controlled MRI scanning room to equilibrate to room temperature. All imaging is performed at the ambient air temperature of 21° C. The scanner used is a General Electric 1.5 T combined with a standard head coil. Imaging parameters are as follows: matrix size 1024×1024, slice thickness 5 mm, TR 3.5 s, Short TEs 30 and 40 ms, long TEs 80, 100, 120, 140, 160, 180 ms using a 32-echo pulse sequence, and 130 Hz/pixel band width. The transverse relaxation rate R₂ images are calculated using in-house written Mathlab scripts (The Mathworks, Inc., Natick, USA), R₂ are calculated as follows:

$$R 2 = \frac{1}{TE 2 - TE 1} \cdot ln ( \frac{S ( TE 1 )}{S ( TE 2 )} ) \qquad ( Eq 1 )$$

Where TE1 is the short TE and TE2 is the long TE and S(TE) is the signal at any given TE. CT plan of the gel phantom is acquired using a spiral CT scanner (Philips CT-Big Bore, Philips Healthcare) in conjunction with the treatment planning system (Eclipse, Varian Medical Systems, Palo Alto, Calif., USA) to generate RapidArc plan (FIG. 2 (Left).

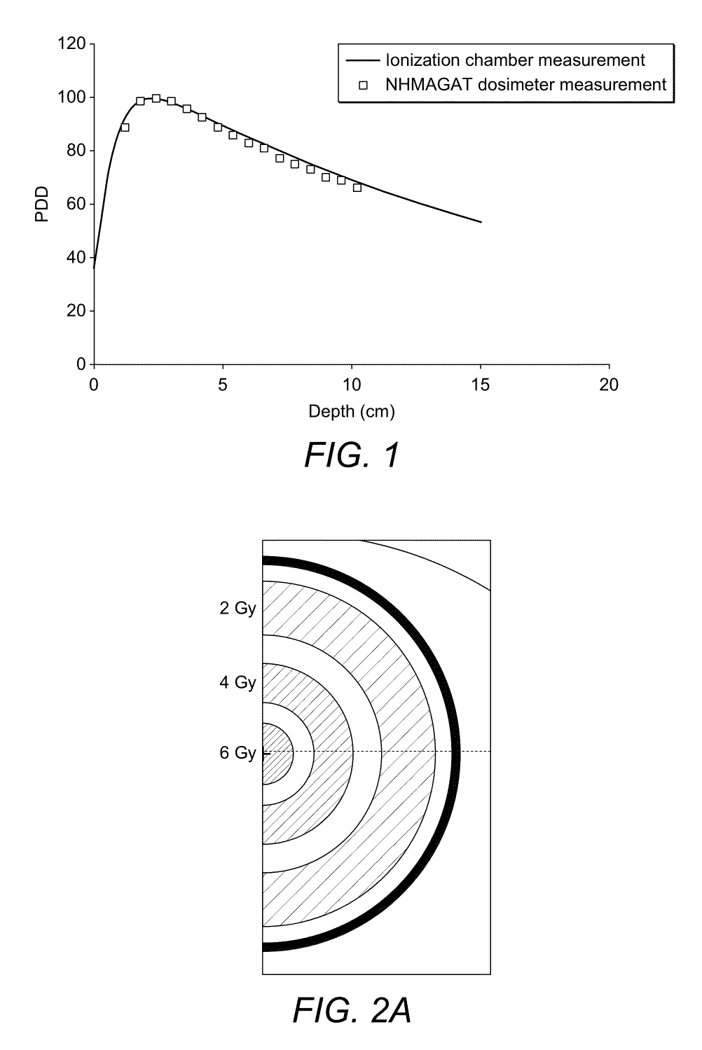

Percentage depth dose (PDD) is defined as the quotient of the absorbed dose at any depth x to the absorbed dose at a fixed reference depth xₘ, along the central axis of the beam (ICRU 1976. Determination of absorbed dose in a patient irradiated by beams of X or gamma rays in radiotherapy procedures. Report 24) or

$$P D D = \frac{{D}_{x}}{{D}_{{x}_{m}}} \times 100 \qquad ( Eq 2 )$$

Where xₘ is the depth of the maximum dose. Percentage depth dose inside bottle (5 cm diameter, 11 cm height) containing NHMAGAT polymer gel dosimeter obtained using equation 1 and equation 2 (Eq 1 and Eq 2) (square) as a function of depth for 8 Gy beam source compared with the measurements by ionization chamber (full curve) is shown in FIG. 1. The deviation between ionization chamber data and NHMAGAT polymer gel data is less than 5%. A 2D dose distribution (axial plane) inside container (12.5 cm diameter, 17 cm height) containing NHMAGAT polymer gel dosimeters obtained using Eq1 (right) are in a good agreement with CT planned dose distribution (left) at the same depth in gel as shown in FIG. 2. The results presented in FIG. 1 and FIG. 2 indicates that NHMAGAT polymer gel dosimeter could be used for radiotherapy treatment planning. Where xₘ is the depth of the maximum dose. Percentage depth dose inside bottle (5 cm diameter, 11 cm height) containing NHMAGAT polymer gel dosimeter obtained using equation 1 and equation 2 (Eq1 and Eq. 2) (square) as a function of depth for 8 Gy beam source compared with the measurements by ionization chamber (full curve) is shown in L The deviation between ionization chamber data and NHMAGAT polymer gel data is less than 5%. A 2D dose distribution (axial plane)inside container(12.5 cm diameter, 17 cm height) containing NHMAGAT polymer gel dosimeters obtained using Eq1 (see FIG. 2(a)) are in a good agreement with CT planned dose distribution (see FIG. 2(b)) at the same depth in gel. The results presented in FIG. 1 and FIG. 2 indicates that NHMAGAT polymer gel dosimeter could be used for radiotherapy treatment planning.

In addition, it will be appreciated that the novel polymer for dosimetric use disclosed herein may be embodied using means for achieving better quality images for medical use and diagnosis. Accordingly, the specification and drawings are to be regarded in an illustrative rather than a restrictive sense.

What is claimed is:

1. A method, comprising:
exposing a specific polymer gel made up of specific components and stored in a specific container to a specific radiation level; and
calculating using a specific equation (equation 1 and 2) to determine a percent depth during a radiotherapy treatment planning using Magnetic Resonance Imaging (MRI) for a patient.

2. The method of claim 1, wherein the specific polymer gel is a normoxic N-(Hydroxymethyl) acrylamide (NHMAGAT) polymer gel dosimeter made up of specific components.

3. The method of claim 2, wherein the specific components are 1-12% w/w % N-(Hydroxymethyl) acrylamide (NHMA) monomer, 1-4% N, N -methylene-bis-acrylamide (BIS) cross-linker, 2-5 g w/w % gelatin (Type A, bloom 300), 0-0.1 mM hydroquinone (HQ), 1-50 mM tetrakis (hydroxymethyl) phosphonium chloride (THPC) and an ultra-pure de-ionized water.

4. The method of claim 3, wherein the specific components are 8 w/w % NHMA monomer, 3 w/w % BIS cross-linker, 3 w/w % gelatin, 0.02 mM HQ, 5 mM THPC and the ultra-pure de-ionized water.

5. The method of claim 1, wherein the specific container is at least one of a tube, bottle and container.

6. The method of claim 1, wherein the specific radiation level is at least one of a 2, 4, 6, 8 and 10 Gy.

7. The method of claim 6, wherein the specific radiation level is 8 Gy.

8. A method, comprising:
adding a gelatin to an ultra pure de-ionized water to soak for 10 minutes to make a mixture;
heating the mixture to 50° C. for 1 hour to make a solution;
cooling the solution down to 40° C. and adding a NHMA, HQ and BIS of specific weight ratio;
stirring the solution containing the NHMA, HQ and BIS to completely dissolve and cool down to 35° C.;
adding a THPC as antioxidant to the solution containing NHMA and HQ;
pouring the solution containing NHMA, HQ and THPC into a specific container; and
forming a N-(Hydroxymethyl) acrylamide (NHMAGAT) polymer gel dosimeter and storing in refrigerator at 10° C. until further use.

9. The method of claim 8, wherein the specific container is at least one of a tube, bottle and container.

10. The method of claim 8, wherein 2-5 g w/w % gelatin and an ultra-pure de-ionized water, 1-12% w/w % N-(Hydroxymethyl) acrylamide (NHMA) monomer, 1-4% N, N -methylene-bis-acrylamide (BIS) cross-linker, 0-0.1 mM hydroquinone (HQ), and 1-50 mM tetrakis (hydroxymethyl) phosphonium chloride (THPC) is added.

11. The method of claim 10, wherein the 8 w/w % NHMA monomer, 3 w/w % BIS cross-linker, 3 w/w % gelatin, 0.02 mM HQ and 5 mM THPC is added.

12. The method of claim 8, further comprising:
exposing the normoxic N-(Hydroxymethyl) acrylamide (NHMAGAT) polymer gel dosimeter made up of specific components and stored in a specific container to a specific radiation level; and
calculating using a specific equation (equation 1 and 2) to determine a percent depth during a radiotherapy treatment planning using Magnetic Resonance Imaging (MRI) for a patient.

13. A method, comprising:
Adding 2-5 g w/w % of a gelatin and an ultra-pure de-ionized water to soak for 10 minutes to make a mixture;
heating the mixture to 50° C. for 1 hour to make a solution;
cooling the solution down to 40° C. and adding a 1-12% w/w % N-(Hydroxymethyl) acrylamide (NHMA) monomer, 0-0.1 mM hydroquinone (HQ), 1-4% N, N -methylene-bis-acrylamide (BIS) cross-linker;
stirring the solution containing the NHMA, HQ and BIS to completely dissolve and cool down to 35° C.;
adding 1-50 mM tetrakis (hydroxymethyl) phosphonium chloride (THPC) to the solution with NHMA, HQ and BIS;
pouring the solution containing NHMA, HQ and THPC into a specific container;
forming a N-(Hydroxymethyl) acrylamide (NHMAGAT) polymer gel dosimeter and storing in refrigerator at 10° C. until further use;
exposing the normoxic N-(Hydroxymethyl) acrylamide (NHMAGAT) polymer gel dosimeter made up of specific components and stored in a specific container to a specific radiation level; and
calculating using a specific equation (equation 1 and 2) to determine a percent depth during a radiotherapy treatment planning using Magnetic Resonance Imaging (MRI) for a patient.

14. The method of claim 13, wherein the specific container is at least one of a tube, bottle and container.

15. The method of claim 13, wherein the specific radiation level is at least one of a 2, 4, 6, 8 and 10 Gy.

16. The method of claim 13, wherein the specific radiation level is 8 Gy.
